(19) United States
(12) Patent Application Publication
(10) Pub. No.: US 2016/0183797 A1
(43) Pub. Date: Jun. 30, 2016

(54) Multifunction Biotelemetry Support System for Psychophysiology Monitoring

(71) Applicant: Czech Technical University in Prague, Faculty of Biomedical Engineering, 272012 Kladno (CZ)

(72) Inventors: Zdenek HON, Holubice (CZ); Pavel SMRCKA, Praha (CZ); Karel HANA, Svetice - R.icany u Prahy (CZ); Jan KASPAR, Jicin (CZ); Marketa JANATOVA, Praha (CZ); Martin VITEZNIK, Praha (CZ); Tomas VESELY, Vysoky Ujezd (CZ); Jan MUZIK, Praha (CZ)

(73) Assignee: Czech Technical University in Prague, Faculty of Biomedical Engineering, 272012 Kladno (CZ)

(21) Appl. No.: 14/953,732

(22) Filed: Nov. 30, 2015

(30) Foreign Application Priority Data
Dec. 31, 2014 (CZ) PV 2014-979

(51) Int. Cl. A61B 5/00; A61B 5/0205; A61B 5/0402

(52) U.S. Cl. CPC A61B 5/0022; A61B 5/0402; A61B 5/0205; A61B 5/01

(57) ABSTRACT

The invention regards Multifunction Biotelemetry Support System for Psychophysiology Monitoring measuring physiological values which are further evaluated, characterized by containing at least one translation unit for WiFi access points and one control unit. Each sensory unit includes an EKG sensor, GSR sensor, RR and BPM sensor, body temperature sensor, room temperature sensor, actigraph activity sensor, HRV measurement and evaluation sensor, signal LED and a push button.

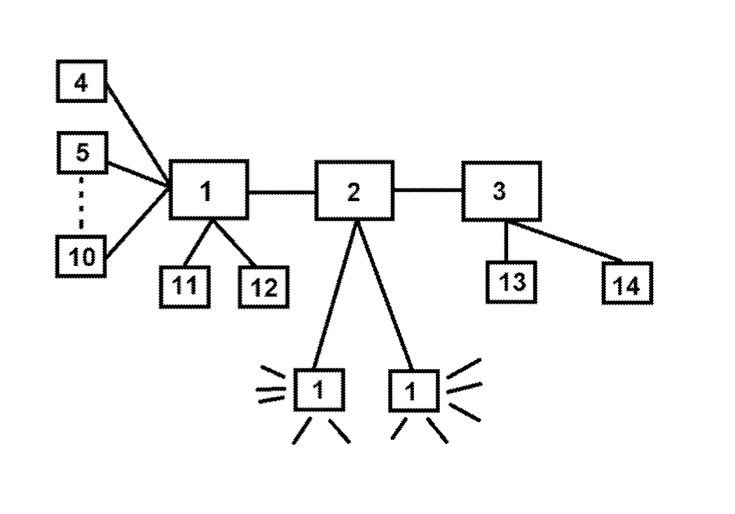

### TECH SECTION

The invention presents a device for online monitoring and evaluation of human psychophysiological state. The device has been designed to monitor subjects e.g. during medical or psychological experiments. The systems allows for the examination of a characteristic response of the organism to stress situations such as mental and emotional pressure or visual, acoustic and other sensory stimuli.

### RECENT STATE OF TECHNOLOGY

Measuring networks comprising several commercially available components is currently in use for monitoring human psychophysiological state: conventional medical sensors such as disposable adhesive electrodes or universal desktop polygraph systems equipped with input/output units for nonstandard variables together with one or more PC-type control units. There is also a specialized and simplified version used in criminalistics, the so-called “lie detector”, which is however a very specialized and single-purpose device and therefore inapplicable for continuous observation of psychophysiological states.

The disadvantage of the current heterogeneous laboratory solution is the difficulty of ensuring signal and data compatibility and the synchronization of individual components together with difficult configurability; from the technical point of view it is an unsystematic solution—a small change in the configuration can reflect in an additional chain of required modifications in other segments of the entire system. Other issues include the low expandability of the system and complicated synchronization with other devices—in case e.g. a medical software, primarily designed for scanning, does not support connection and operation of a stimulation unit or other modality requiring data communication with external software as it is impossible to incorporate these modules into the system

Current solutions in the commonly available setup similarly do not allow for real-time evaluation or classification of psychophysiological states online as the control software, which is primarily designed for diagnostics lacks appropriate design for such substantially different tasks as real-time evaluation.

Another disadvantage of current systems is their dimensions and laboratory arrangement which does not allow for their portability—these typically comprise of a set of desktop devices mounted to the laboratory interior.

An outstanding shortcoming of current possibilities is also the classic cable wiring of a desktop sensory unit with PC control unit—the examined person has to either sit or lie during the experiments and cannot freely move within the room where the experiment is taking place.

Considerable costs of construction of the measuring network, which has to be composed of several universal medical devices, pose yet another disadvantage as it totals thousands of dollars. Since the accuracy of psychophysiological measurements using current devices is greater than necessary, practical use of current systems proves uneconomical. High sensitivity of current systems and demanding technical skills for their operation and maintenance contribute to their disadvantage due to their networking nature requiring trained personnel to secure reliable application of sensors to bodies of subjects and calibration of the systems to guarantee relevance of the measured data.

### THE ESSENCE OF THE DEVICE

The above disadvantages are overcome to a great extent by the Multifunction Biotelemetry Support System for Psychophysiology Monitoring measuring and further evaluating physiology variables in real time. The core feature is the presence of at least one sensory unit equipped with at least one translation unit for WiFi access points and one control unit. Each sensory unit includes an EKG sensor, GSR sensor, RR and BPM sensor, body temperature sensor, room temperature sensor, actigraph activity sensor, HRV measurement and evaluation sensor, signal LED and a push button.

The control unit is equipped with programmable device for analyzing input data and which case of a change in this data compared to long-term status uses the feedback module to inform the family of the user and/or a psychotherapist/psychiatrist about the change in the mental state of the user with the aim to prevent consequent changes in the state of the user.

The control unit consists of a desktop PC, a notebook or a tablet.

The sensory unit is converted into a pocket-size, mechanically resilient form, which uses wireless interface (Bluetooth, WiFi, ZigBee or other) for communication with the control computer. This enables the monitored person to move freely within the range of the wireless interface. Furthermore the whole system is made portable without any additional setup requirements and ready for application in field when battery powered.

The advantage of aforementioned solution is the possibility to integrate input sensors into a flexible thoracic harness and/or a special sensory suit retaining the possibility of mounting sensors using classic disposable sensors, e.g. Ag—AgCl EKG sensors. This considerably facilitates the application of the device in practice, reduces the requirements for its maintenance, and enhances the quality of measured signals as for e.g. higher resistance against movement artifacts.

The feature of incorporating all input/output signals into a single connector presents yet another advantage of the system, allowing for the system to turn on, calibrate, connect with the thoracic unit and start monitoring without further configuration by simply plugging the connector into the wireless sensory unit.

The device is therefore comprised of input sensors according to required configuration such as EKG, GSR, temperature sensors etc., optional output stimulation units such as optical or acoustic, pocket wireless sensory unit and a control PC unit equipped with special software ensuring cooperation of all components, autocalibration and receptor checks, course record, online classification and continuous visualization of psychophysiological state.

The device relies on the principle of noninvasive surface measurement of physiological values, their wireless transfer and intelligent computer assessment resulting in continuous estimation of the extent of mental pressure or evaluation of the intensity of the reaction to individual impulses using custom developed built-in algorithms.

Owing to the described technical solution to the device is more mechanically resilient than a measuring network comprising several commonly available desktop medical devices.

The described technical solution results in a much more affordable system if considering procurement, operating, as well as maintenance costs, together with costs for staff training, while maintaining accuracy of the results.

The communication is provided for by a wireless interface transferring a full stream of data. WiFi interface including data roaming is used (optionally Bluetooth or Xbee), enabling coverage of larger areas. A remote access to the Internet is also available, isolating the control of the experiment by the visualization unit from the subject's environment.

The system offers simultaneous data saving on a memory (e.g. SD) card during wireless transfer as well as during loss of connection with the visualization unit. The solution has the ability to work independently even in the case of loss of wireless connection with the visualization unit relying on the data card record and subsequent synchronization.

The option of simultaneous monitoring of several subjects—several to several dozen—also adds to the advantage of the system.

The solution provides for automatic evaluation of the psychophysiological state of a subject and/or the state of mental resistance against—the extent of stress and the intensity of the reaction and/or the speed of their response to presented stimuli.

Another advantage is the possibility of continuous monitoring either within a short-term period or up to long-term horizon ranging from several to dozens of hours.

There is also the option of repeated measurement over a period of days, months or years. The long-term monitoring mode relies on the control unit and the programmable device module responsible for continuous analysis of input data—the number of text messages sent by the user, the number of contacts or posts on social networks. It evaluates these figures and in case of change in these numbers or change in the trend compared to the previous course informs the family of the user and/or a psychotherapist/psychiatrist about the change in the mental state of the user with the aim to prevent consequent changes in the state of the user.

### DESCRIPTION OF THE TECHNICAL DRAWING

Multifunction biotelemetry system will be described in detail demonstrating a particular application, which is presented by the drawing.

### EXAMPLE OF THE INVENTION APPLICATION

Multifunction Biotelemetry Support System for Psychophysiology Monitoring of psychophysiological state in real time, measuring physiological values which are further evaluated uses at least one sensory unit equipped with at least 1 translation unit for 2 WiFi access points and one 3 control unit. Each 1 sensory unit includes an 4 EKG sensor, 5 GSR sensor, 6 RR and BPM sensor, 7 body temperature sensor, 8 room temperature sensor, 9 actigraph activity sensor, 10 HRV measurement and evaluation sensor, 11 signal LED and 12 push button.

The 3 control unit is equipped with 13 programmable device for analyzing input data and which case of a change in this data compared to long-term status uses the 14 feedback module to inform the family of the user and/or a psychotherapist/psychiatrist about the change in the mental state of the user with the aim to prevent consequent changes in the state of the user. The control unit is a 3 tablet.

As illustrated by the attached technical drawing, the system consists of the following parts: sensors 4-10—these are biocompatible surface sensors of physiological values, typically EKG electrodes, GSR sensors, body temperature, push button etc. These can be set up in a classic medical fashion, e.g. in the form of AgCl EKG electrodes and/or can made a part of the flexible thoracic harness and/or the sensory tricot for greater advantage.

Components of the device also include optional output units—within common configuration represented by 11 signal LED and 12 push button. Short wiring (up to 20 inches) on the subject's body is used to connect sensors 4 and 10 to the wireless 1 sensory unit. This arrangement allows the subject to move freely within the laboratory premises.

Several 1 units wired to a set 4-10 of sensors, 11 signal LED and 12 push button are connected to the 3 control unit using the 2 wireless infrastructure. Each 1 unit and the set of sensors is mounted on the subject's body confronted with physical stimuli, or stimuli generated by the 3 control unit supported by 11 signal LED in the form of visual and/or other stimuli. Using 4-10 sensors and/or 12 the push button, subject's reactions are collected, and then stored, visualized and evaluated in 3 control unit for the purpose of assessing the psychophysiological state, or the status of mental resistance against the presented stimuli—the extent of the stress triggered and the reaction and/or the speed of the subject's reaction to the stimuli.

This procedure may be used for continuous monitoring either within a short-term period or up to long-term horizon ranging from several to dozens of hours.

There is also the option of repeated measurement over a period of days, months or years. The long-term monitoring mode relies on the 3 control unit and the 13 programmable device module responsible for continuous analysis of input data—the number of text messages sent by the user, the number of contacts or posts on social networks. It evaluates these figures and in case of change in these numbers or a change compared to long-term status uses the 14 feedback module to inform the family of the user and/or a psychotherapist/psychiatrist about the change in the mental state of the user with the aim to prevent consequent changes in the state of the user e.g. in the field of psychology or psychiatry to monitor patients with mental disorders schizophrenia, bipolar disorder etc.

Wireless sensory unit includes modules of measuring amplifiers for individual input variables, output units interface, central processing unit with (typically 16-bit) A/D converter and sampling frequency ranging from 200 to 600 Hz, and a wireless communication interface unit usually represented by Bluetooth device with the range of approx. 82 feet.

Typical parameters of the described example are as follows:
EKG: range 6 mV, frequency range 0.05450 Hz, input resistance: 10 Mohm, CMRR 104 dB
GSR: range 0-1,000,000 Ohm, accuracy: 10%
Temperature: range: 95-113° F., accuracy +/−0.05° F., time step 0.1 s
Signalization: manual push button
Optical signalization: high luminous density LED
Digital output: “on” mode: 3.3 V max, “off” mode: lower than 0.2 V
Power supply: built-in LiPol battery, approx. 10 hours of continuous operation without charging when using on-line data transmission, approx. 2 days of continuous operation without charging when using memory card. Charging time approx. 4 hours
Built in timer/clock backed up by a lithium battery
Parameters of communication interface of the sensory unit for real time streaming of the measured biosignals:
   WiFi technology: sensory unit functioning as a server, to which the control unit connects using the software and further communicates using BSD sockets (requiring IP address of the sensory unit, communication port number and possible data encryption key).
   Communication may be either open (open system) or encrypted using WEP-128, or WPA-PSK/WPA2-PSK.
   Typical network typology of the used wireless network: sensory units connected to WiFi access point according to the IEEEE802.11g standard, which works as a router and allows up to 54 Mbit/s (typically 20 Mbit/s) connection over a range of approx. 130 feet, up to 490 feet in open space. Sensory units may have static or assigned IP address assigned by DHCP server of the WiFi access point. Control computer typically connects to the WiFi access point using standard Ethernet cable. If necessary, larger areas may be covered by interconnecting several WiFi access points.
Control microprocessor of the sensory unit: ARM Cortex M3 (LPC1769) cure microprocessor frequency (depending the operation mode, up to 120 MHz)
   System requirements: desktop PC and/or notebook or tablet supporting MS Windows 7 and/or Android 4. Sufficient free space of the memory card or HDD, ranging in hundreds of MBs, WiFi or Ethernet interface.
   Ability to recognize connected units, real time-visualization of signals, communication breakdowns detection and automatic recovery
   Ability to incorporate algorithms for data analysis using plug-in form (HRV analysis module, physical and mental pressure analysis module)

The above parameters relate to the particular example of application, however the system may be custom modified to employ required input/output units and respective plug-ins and software for further or nonstandard data processing and visualization. The system also includes control computer equipped with special control program allowing for simultaneous recording of all observed parameters on HDD, online preview of the measured curves, inserting time markers, real time estimation-classification of psychophysiological state and continuous visualization of results.

The solution offers recording the stream of the measured data on memory card. The maximum record length depends on the number of measured channels and selected sampling frequency. Using sampling frequency of 500 Hz and simultaneous employment of four 16 bit channels allows for a record as long as 5 consecutive days.

### INDUSTRIAL USE

The device may be used to examine reactions of human organism to stimuli, which are assumed to trigger typical response in common biosignals, detectable on the body surface. Typical use represents assessment of reaction type to various form of mental or emotional pressure during medical or psychological experiments, i.e. so estimation of the so called psychophysiological state. Further possibility of the system's use is evaluation of reactions to stress stimuli during the course of training or actual work performance of technical device operators at positions related to power plants, aviation, or any other where monitoring of psychophysiological state is advisable.

1. A biotelemetry support system for psychophysiology monitoring in real time, measuring physiological values which are further evaluated, comprising
at least translation unit for WiFi access points and one control unit,
at least one sensory unit including an EKG sensor, a GSR sensor, a RR and BPM sensor, a body temperature sensor, a room temperature sensor, an actigraph sensor, an HRV measurement and evaluation sensor, a signal LED, and a push button.

2. The biotelemetry system according to claim 1, wherein the control unit is equipped with a programmable device for analyzing input data of a subject and which, in case of a change in the input data compared to long-term status, uses a feedback module to inform an observer about a change in a mental state of the subject with an aim of preventing consequent changes in a state of the subject.

3. The biotelemetry system according to claim 2, wherein the control unit comprises a desktop PC, notebook or tablet.

4. The biotelemetry system according to claim 1, wherein the control unit comprises a desktop PC, notebook or tablet.
